(19) United States
(12) Patent Application Publication
(10) Pub. No.: US 2021/0360929 A1
(43) Pub. Date: Nov. 25, 2021

(54) Quantum Dots for Fungicidal Applications and Methods of Use

(71) Applicant: Board of Regents, the University of Texas System, Austin, TX (US)

(72) Inventors: Juan Adrian Zepeda-Galvez, San Antonio, TX (US); Janeth Alexandria Garcia-Monge, San Antonio, TX (US); Clarissa Vazquez-Colon, San Antonio, TX (US); Alvaro Flores-Pacheco, San Antonio, TX (US); Humberto H. Lara-Villegas, San Antonio, TX (US); Arturo A. Ayon, San Antonio, TX (US)

(21) Appl. No.: 17/330,335

(22) Filed: May 25, 2021

Related U.S. Application Data
(60) Provisional application No. 63/029,586, filed on May 25, 2020.

(51) Int. Cl. A01N 59/16; A01P 3/00; A01N 59/00; A01N 25/08; A61L 29/10; A61L 29/16; A61L 27/30; A61L 27/54; A61L 31/08; A61L 31/16

(52) U.S. Cl. CPC A01N 59/16; A61L 2300/404; A01N 59/00; A01N 25/08; A61L 29/106; A61L 29/103; A61L 29/16; A61L 27/306; A61L 27/303; A61L 27/54; A61L 31/088; A61L 31/084; A61L 31/16; A61L 2300/102; A61L 2300/10; A61L 2300/108; A61L 2430/20; A61L 2430/24; A01P 3/00

(57) ABSTRACT

Certain embodiments are directed to methods and compositions for inhibiting, stabilizing or preventing fungal infections by yeast on a surface using an agent comprising one or more types of quantum dots sufficient to regulate the growth of fungal cells or biofilms thereof.

### PRIORITY

This application claims priority to U.S. Provisional Application Ser. No. 63/029,586 filed May 25, 2020, which is incorporated herein by reference in its entirety.

### STATEMENT REGARDING FEDERALLY FUNDED RESEARCH

This invention was made with government support under FA9550-17RYCOR490 awarded by the Air Force Office of Scientific Research. The government has certain rights in the invention.

### TECHNICAL FIELD

The present invention relates generally to the fields of cell and molecular biology, nanomedicine, and the treatment of fungal infections. In particular, the invention concerns the use of quantum dots as antifungal agents to inhibit, stabilize, or prevent fungal infections by yeast.

### BACKGROUND

Candida auris (C. auris) is an emergent opportunistic fungal pathogen, originally isolated in Japan in 2009.¹ There have been several recent outbreaks of C. auris infections with high morbidity and mortality rates in healthcare centers around the world.²⁻⁶ The actual number and severity of the outbreaks, however, has likely been underreported, which can be attributed to the difficulty of identifying C. auris due to a lack of suitable laboratory instrumentation, especially in developing countries.⁷

C. auris is the first yeast to be categorized as a public health threat owing to (i) multi-drug resistance (MDR) characteristics of the yeast, including resistance to azoles; (ii) the ineffectiveness of human neutrophils in eliminating C. auris infection^{8,9}; (iii) the ability of the yeast to colonize skin and cause severe diseases; and (iv) the high mortality rates associated with C. auris infection. Additionally, the yeast can form a biofilm on biomedical instruments and other surfaces in healthcare facilities. Infections associated with the biofilm growth usually are challenging to eradicate because biofilm microbes can share nutrients and are sheltered from harmful environment factors, including desiccation, drugs, and the host's immune system. Further, the fight against drug-resistant bacteria and bacteria-based illnesses is hampered by the fact that bacteria tend to be coated with a fungus layer than inhibits or even totally precludes the beneficial effects of antibiotics.

Invasive candidiasis produced by C. auris has become a major health complication due to resistance of the yeast to antifungals primarily used to treat yeast infections, including fluconazole and even commercially available wipe-based disinfectants.^{13,14} In fact, the Center for Disease Control (CDC) has indicated that C. auris is infrequently eliminated by commonly used antifungal medications, and, as a consequence, C. auris infections are difficult to treated. Although echinocandins have been recommended by the CDC for treating C. auris infections, this is a temporary solution as FKS genes, known to cause resistance to echinocandins, have been found in the C. auris genome. Furthermore, a few C. auris isolates have been found to be resistant to all classes of antifungal agents, even amphotericin B.¹⁵

Several studies support the notion that surface contamination in healthcare facilities and medical devices are the main sources of yeast transmission. ^{10,16} Furthermore, it has been shown that C. auris can survive on a variety of surfaces for a prolonged time, and the yeast can form biofilms in both dry and humid environments.^{10,11} It has also been observed that patients that have undergone medical interventions and have an indwelling venous catheter or other biomedical instrument entering the body are more likely to be infected by C. auris.¹²

Fungicides can have an enormous effect in the prevention and fight against various diseases and its spread in hospital settings. Because of the high rates of resistance of preformed C. auris biofilms to antifungal drugs, there is an urgent need to develop effective fungicidal options to treat fungal infections, including new and efficacious surface treatments to preclude proliferation of fungal microbes in healthcare facilities.

### SUMMARY

Despite their potential efficacy as antifungal and antimicrobial agents, quantum dots (QDs) have been studied only sporadically with respect to treatment of fungal infections, and such studies have been limited to metal-based and carbon-based QDs.¹⁷⁻¹⁹ Quantum dots (QDs) are man-made nanoscale crystals that that can transport electrons. Certain aspects of the present disclosure are directed to establishing QD-based treatments for fungal infections to inhibit, stabilize, or prevent microbe growth on invasive biomedical instruments and in healthcare facilities. A further aspect relates to use of functionalized surfaces coated with QDs.

The inventors discovered a solution to address the problems associated with fungal infection. The solution resides in inhibition of fungal cell growth and/or biofilm formation by QDs. In particular, applications of zinc oxide, silicon, zinc sulfide, and carbon QDs inhibited biofilm formation by C. auris, effectively treating or ameliorating infection and/or contamination by the yeast.

Certain embodiments are directed to methods for treating a microbial infection, microbial contamination, biofilm, or growth on a surface, the method comprising applying to a surface in need of such treatment or a surface at risk of exposure to microbes a composition comprising an effective amount of one or more quantum dot species. In certain aspects, the effective amount of the one or more quantum dot species comprises an amount effective to inhibit, stabilize, or prevent growth of fungal cells or formation of biofilms. In certain aspects, the inhibition by the one or more quantum dot species reduces growth of fungal cells or formation of biofilms by greater than or equal to about 50% compared to the growth of fungal cells or biofilms that have not been exposed to the one or more quantum dot species. In a further aspect, the inhibition by the one or more quantum dot species reduces growth of fungal cells or formation of biofilms by greater than or equal to about 70% compared to the growth of fungal cells or biofilms that have not been exposed to the one or more quantum dot species. The quantum dots can have a maximum dimension of less than or equal to about 5 to 20 nm. In certain aspects, the quantum dots have a maximum dimension of less than or equal to about 5 to 10 nm. In a further aspect, the one or more quantum dot species comprise a quantum dot comprised of a main group IV element. In a particular aspect, the main group IV element comprises silicon or carbon. The one or more quantum dot species can be a quantum dot comprising an alloy. The alloy can be a transition metal group II element and a main group VI element. In certain aspects, the transition metal group II element is zinc or cadmium. In a further aspect, the main group VI element comprises oxygen, sulphur, selenium, or tellurium. In certain aspects, the concentration of the one or more quantum dot species applied is between about 10 μg/mL and about 200 μg/mL or about 1 to 20 μg/cm². In a further aspect, the concentration of the one or more quantum dot species applied is between about 25 μg/mL and about 150 μg/mL or 2.5 to 15 μg/cm².

The microbial infection can be a fungal infection caused by yeast. In certain aspects, the yeast is the genus Candida. In a further aspect, the yeast is C. glabrata, C. auris, C. haemulonii, C. guilliiermondii, C. krusei, C. lusitaniae, C. kefyr, Yarrowia (C.) lypolitica, C. rugose, or a combination thereof. In certain aspects, the surface is the surface of an in-dwelling medical device. The in-dwelling medical device can be, but is not limited to a surgical implant, prosthetic device, artificial joint, heart valve, pacemaker, vascular graft, vascular catheter, cerebrospinal fluid shunt, urinary catheter, or continuous ambulatory peritoneal dialysis catheter. In certain aspects, the one or more quantum dot species are applied to the in-dwelling medical device immediately (within 10 minutes or an hour) before insertion. The surface can be a surface of a medical or surgical tools, devices, instruments, implements, and equipment. The method can further include applying the one or more quantum dot species either sequentially or simultaneously with one or more additional therapeutic agents. The one or more additional therapeutic agents can be, but is not limited to an anti-fungal agent or an anti-yeast agent. The anti-fungal or anti-yeast agent can be, but is not limited to an echinocandin, azole, polyene, allylamine, fluorinated pyrimidine analog, or a combination thereof.

Certain embodiments are directed to an antimicrobial material. The antimicrobial material being a bioactive coating comprising an effective amount of the one or more quantum dot species. The microbe can be a fungal yeast. In a particular aspect, the yeast are of the genus Candida. The yeast can be C. glabrata, C. auris, C. haemulonii, C. guilliiermondii, C. krusei, C. lusitaniae, C. kefyr, Yarrowia (C.) lypolitica, C. rugose, or a combination thereof. In certain aspects, the antimicrobial material reduces the growth of fungal cells or formation of biofilms thereof by greater than or equal to about 50% as compared to a surface without the bioactive coating. In certain aspects, the antimicrobial material reduces the growth of fungal cells or formation of biofilms by greater than or equal to about 70% as compared to a surface without the bioactive coating. The quantum dots can have a maximum dimension of less than or equal to about 20 nm (e.g., 0.1 to 20 nm). In further aspects, the quantum dots can have a maximum dimension of less than or equal to about 10 nm (e.g., 0.1 to 10 nm). The one or more quantum dot species can comprise a main group IV element. In certain aspects, the main group IV element includes silicon or carbon. The one or more quantum dot species can include a quantum dot comprising an alloy. The alloy can be a transition metal group II element and/or a main group VI element. The transition metal group II element can be zinc or cadmium. The main group VI element can be oxygen, sulphur, selenium, or tellurium. The antimicrobial material can be a coating of or coat an in-dwelling medical device. The in-dwelling medical device can be a surgical implant, prosthetic device, artificial joint, heart valve, pacemaker, vascular graft, vascular catheter, cerebrospinal fluid shunt, urinary catheter, or continuous ambulatory peritoneal dialysis catheter. The antimicrobial material can be a coated on the surface medical or surgical tools, devices, instruments, implements, and equipment.

Certain embodiments include compositions having one or more quantum dot species, wherein the composition is effective to inhibit, stabilize, or prevent growth of fungal cells or formation of biofilms thereof. The inhibition by the one or more quantum dot species can reduce growth of fungal cells or formation of biofilms by greater than or equal to about 50% (e.g., 50 to 98% or more). The inhibition by the one or more quantum dot species can reduce growth of fungal cells or formation of biofilms by greater than or equal to about 70% (e.g., 70 to 98% or more).

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Throughout this disclosure, numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. By way of example, it may be less than 5% of the value indicated and in certain variations, optionally less than 1% of the value indicated. In addition, disclosure of ranges includes disclosure of all values and further divided ranges within the entire range, including endpoints and sub-ranges given for the ranges.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 shows the dose-response curve for the fungicidal activity of zinc oxide QDs.

FIG. 2 shows the dose-response curve for the fungicidal activity of zinc sulfide QDs.

FIG. 3 shows the dose-response curve for fungicidal activity of silicon QDs.

FIG. 4 shows the dose-response curve for the fungicidal activity of carbon QDs.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### I. QUANTUM DOTS

As used herein, the term “quantum dots” (“QDs”) refers to zero-dimensional nanosemiconductor materials comprising nanocrystals in which so-called quantum effects occur due to their extremely small diameter. Quantum effects cause interesting optical, magnetic, and electronic properties in nanocrystals. For example, QDs can fluoresce with the aid of light, efficiently supply electricity, or serve as a memory or processor elements in information technology. Specifically, QDs are theoretically described as a point-like, or a zero dimensional entity. They are comprised of tiny particles or nanocrystals of a semiconducting material with diameters typically ranging from 1-100 nanometers (5-500 atoms). Most QD properties depend on the dimensions, shape, and materials of which QDs are made. Generally, QDs present different thermodynamic properties from the bulk materials of which they are made. One of these effects is the Melting-point depression. Optical properties of spherical metallic QDs are well described by the Mie scattering theory. QDs also display unique electronic properties, intermediate between those of bulk semiconductors and discrete molecules, that are partly the result of the unusually high surface-to-volume ratios for these particles.

QDs can be made of one or more different materials, which can lead to construction of different species of dots with different structures. Core-type QDs can be single component materials with uniform internal compositions, such as chalcogenides (selenides, sulfides or tellurides) of metals like cadmium, lead or zinc, for example, CdTe or PbS. Core-shell QDs have small regions of one material embedded in another, such as QDs with CdSe in the core and ZnS in the shell. Coating quantum dots with shells improves quantum yield by passivizing nonradiative recombination sites and also makes them more robust to processing conditions for various applications. Often different materials are used for the core and shell, whereby several coating layers are also possible. Both the electronic and optical properties of the quantum dots can be precisely adjusted with core-shell structures. Alloyed quantum dots have both homogeneous and gradient internal structures to achieve continuous tuning of the optical properties without changing the particle size.

Spherical QD particles are usually prepared with atoms from groups II-VI, III-V, or IV-VI in the periodic table, and eventually become many different types of alloys. Specifically, there are three main types of QD alloys: (1) III-V semiconductors, made of elements from the periodic table from main group III (boron, aluminum, gallium, indium) and main group V (nitrogen, phosphorus, arsenic, antimony, bismuth); (2) II-VI semiconductors, made of elements of transition metal group II (zinc, cadmium) and main group VI (oxygen, sulphur, selenium, tellurium); and (3) main group IV (silicon, germanium, carbon, lead) and main group VI elements. Common QD alloys are summarized in Table 1.

TABLE 1
Quantum Dot Alloys

| Type | Quantum Dots |
|---|---|
| II-VI | CdS, CdSe, CdTe, ZnS, ZnO, ZnSe, ZnTe, HgS, HgSe |
| III-V | GaAs, InGaAs, InP, InAs |
| IV-VI | C, Si, Ge, PbS, PbSe |

QDs can be made in several ways, including but not limited to colloidal synthesis, plasma synthesis, self-assembly, and electrochemical assembly. Colloidal synthesis involves heating solutions at a high temperature, causing elemental precursors to decompose to monomers, which nucleate and generate nanocrystals. Temperature is a critical factor in colloidal synthesis of QDs and must be high enough to allow for rearrangement and annealing of atoms but low enough to promote crystal growth. The concentration of monomers is also important and must be stringently controlled during nanocrystal growth because the growth process of nanocrystals occurs in “focusing” and “defocusing” regimes. At high monomer concentrations, the critical size at which nanocrystals neither grow nor shrink is relatively small, resulting in growth of nearly all particles in a “focusing” regime. In this regime, smaller particles grow faster than large ones because larger crystals need more atoms to grow than small crystals, resulting in “focusing” of the size distribution and yielding an improbable distribution of nearly monodispersed particles. Size focusing is optimal when the monomer concentration is kept such that the average nanocrystal size present is always slightly larger than the critical size. Over time, the monomer concentration diminishes, the critical size becomes larger than the average size present, and the distribution “defocuses.”

There are colloidal methods to produce many different QD species. Typical QDs are made of binary compounds such as lead sulfide, lead selenide, cadmium selenide, cadmium sulfide, cadmium telluride, indium arsenide, and indium phosphide. QDs may also be made from ternary compounds such as cadmium selenide sulfide. Further, recent advances have been made which allow for synthesis of colloidal perovskite QDs. These QDs can contain as few as 100 to 100,000 atoms within the QDs volume, with a diameter of ≈10 to 50 atoms. This corresponds to about 2 to 10 nanometers, 10 nm in diameter. Further, large batches of QDs may be synthesized via colloidal synthesis. Due to this scalability and the convenience of benchtop conditions, colloidal synthetic methods are promising for commercial applications.

Plasma synthesis is a gas-phase approach for synthesizing QDs and is often used to produce QDs having covalent bonds. The size, shape, surface, and composition of QDs can all be controlled in non-thermal plasma. Plasma synthesis has also improved doping of QDs, or adding impurities to the nanocrystals forming QDs to alter the electronic properties of the QDs. QDs synthesized by plasma are usually in the form of powder, for which surface modification may be carried out. This can lead to excellent dispersion of QDs in either organic solvents or water.

Highly ordered arrays of QDs may also be self-assembled by electrochemical techniques. A template is created by causing an ionic reaction at an electrolyte-metal interface which results in the spontaneous assembly of nanostructures, including QDs, onto the metal which is then used as a mask for mesa-etching these nanostructures on a chosen substrate. In electron beam lithography, for example, QDs are “written” onto an appropriate substrate via an electron beam and then exposed by a suitable etching process. In brief, a special lacquer is applied to a surface, which contains the components for the generation of the desired QDs. The spot-like electron beam converts the components into QDs at the very small points where it hits the lacquer surface. The excess paint residues are then removed.

QDs between 5 and 50 nm in size can also self-assemble. Self-assembled QDs nucleate spontaneously under certain conditions during molecular beam epitaxy (MBE) and metalorganic vapor-phase epitaxy (MOVPE), when a material is grown on a substrate to which it is not lattice matched. The resulting strain leads to the formation of islands on top of a two-dimensional wetting layer. This growth mode is known as Stranski-Krastanov growth. The islands can be subsequently buried to form the QD. A widely used type of QDs synthesized with this method are In(Ga)As QDs. Such QDs have the potential for applications in quantum cryptography (i.e. single photon sources) and quantum computation.

Complementary metal-oxide-semiconductor (CMOS) technology can be employed to fabricate silicon QDs. Ultra-small (L=20 nm, W=20 nm) CMOS transistors behave as single electron QDs when operated at cryogenic temperatures over a range of about −269° C. (4 K) to about −258° C. (15 K). The transistor displays Coulomb blockade due to progressive charging of electrons one by one. The number of electrons confined in the channel is driven by the gate voltage, starting from an occupation of zero electrons, and it can be set to one or many.

In some embodiments, the present disclosure provides one or more QD species capable of use as an antimicrobial agent. In some embodiments, the antimicrobial agent is an antifungal agent. In certain variations, the QD species comprises a metal oxide material. In certain aspects, the metal is zinc and the metal oxide is zinc oxide. In certain aspects, the metal oxide is zinc sulfide. In other variations, the QD species comprises silicon. In other variations, the QD species comprises carbon.

The QDs are generally less than about 1 μm (i.e., 1,000 nm). A QD generally refers to a nano-component where all three spatial dimensions are nano-sized and less than or equal to a micrometer (e.g., less than about 1,000 nm). In accordance with the present disclosure, a QD may have at least one spatial dimension that is less than or equal to about 100 nm, less than or equal to about 90 nm, less than or equal to about 80 nm, less than or equal to about 70 nm, less than or equal to about 60 nm, less than or equal to about 50 nm, less than or equal to about 40 nm, less than or equal to about 30 nm, less than or equal to about 25 nm, less than or equal to about 20 nm, less than or equal to about 15 nm, less than or equal to about 10 nm, less than or equal to about 5 nm, and in certain variations, less than or equal to about 4 nm, less than or equal to about 3 nm, less than or equal to about 2 nm, less than or equal to about 1 nm, or less than or equal to about 0.1 nm. In certain embodiments, the QD has at least one spatial dimension that is greater than or equal to about 0.1 nm to less than or equal to about 50 nm, greater than or equal to about 0.1 nm to less than or equal to about 25 nm, greater than or equal to about 0.1 nm to less than or equal to about 20 nm, greater than or equal to about 0.1 nm to less than or equal to about 15 nm, greater than or equal to about 0.1 nm to less than or equal to about 10 nm, and in certain variations, greater than or equal to about 0.1 nm to less than or equal to about 5 nm.

In certain embodiments of the present disclosure, a QD has all three spatial dimensions that are less than or equal to about 100 nm, less than or equal to about 90 nm, less than or equal to about 80 nm, less than or equal to about 70 nm, less than or equal to about 60 nm, less than or equal to about 50 nm, less than or equal to about 40 nm, less than or equal to about 30 nm, less than or equal to about 25 nm, less than or equal to about 20 nm, less than or equal to about 15 nm, less than or equal to about 10 nm, less than or equal to about 5 nm, and in certain variations, less than or equal to about 4 nm, less than or equal to about 3 nm, less than or equal to about 2 nm, less than or equal to about 1 nm, or less than or equal to about 0.1 nm. In certain other embodiments, all dimensions of the QDs are greater than or equal to about 0.1 nm to less than or equal to about 50 nm, greater than or equal to about 0.1 nm to less than or equal to about 25 nm, greater than or equal to about 0.1 nm to less than or equal to about 20 nm, greater than or equal to about 0.1 nm to less than or equal to about 15 nm, greater than or equal to about 0.1 nm to less than or equal to about 10 nm, and in certain variations, greater than or equal to about 0.1 nm to less than or equal to about 5 nm.

The QDs used in the compositions and methods of the present disclosure can be prepared by any of the methods described herein. In certain embodiments, the QDs comprise zinc oxide, zinc sulfide, silicon, or carbon, though similar effects may be obtained for other species of QDs formed from other materials. Such other materials include, but are not limited to, zinc telluride, zinc selenide, cadmium chalcogenides, manganese oxide, silica oxide, alumina oxide aluminosilicate, metal oxides, and other metals, including gold, silver, platinum, osmium, iridium, ruthenium, rhodium, palladium, aluminum, chromium, cobalt, copper, iron, magnesium, nickel, tantalum, tin, titanium, tungsten, and vanadium. The QD surfaces can also be modified with organic functional substituents, polymers or plastic oligomers, or carbon powder particles.

In various aspects, the present disclosure contemplates the use of QDs as microbial agents. In some embodiments, the microbial agents are fungicidal agents. The benefits of such engineered particles over traditional microbial agents are multi-fold. The QDs are resistant to normal biological degradation processes giving them a long life span. The size and shape of the QD can be controlled to tune the level or extent of microbial inhibition. Antimicrobial activity against multiple microbial targets reduces the potential for the development of tolerance or resistance. Furthermore, the QDs can easily be applied to surfaces or substrates to create bioactive surface coatings.

### II. QUANTUM DOTS FOR INHIBITION OF FUNGAL GROWTH

The present disclosure provides methods and compositions of inhibiting, stabilizing, or preventing fungal infections caused by yeast comprising administering an effective amount of the quantum dots to a surface in need of such a treatment, wherein the yeast is sensitive to the QDs. The method is particularly useful when the yeast is resistant to antifungals other than QDs.

As used herein, the term “fungal infection” refers to an invasion by fungal cells resulting in candidiasis caused by Candida yeast on a surface or within a host. For example, the infection may include the excessive growth of fungi that are normally present in or on the surface or host, or growth of fungi that are not normally present in or on the surface or host. More generally, a fungal infection can be any situation in which the presence of a fungal population is detrimental or damaging. Thus, a surface or host is “suffering” from a fungal infection when an excessive amount of a fungal population is present in or on the surface or host, or when the presence of a fungal population is damaging.

Candida normally lives on the skin and inside the body, in places such as the mouth, throat, gut, and vagina, without causing infection, though some species of Candida can cause infection in people. Infections are usually diagnosed by culture of blood or other body fluids. Candidiasis that develops in the mouth or throat is called thrush or oropharyngeal candidiasis. Candidiasis in the vagina is commonly referred to as a yeast infection. Invasive candidiasis occurs when Candida species enter the bloodstream or affect bones, eyes, and internal organs like the kidney, heart, or brain. Candidemia, a bloodstream infection with Candida, is a common infection in hospitalized patients.

Several classes of antifungal drugs are available to treat severe Candida infections, including azoles, echinocandins, and amphotericin B. Some types of Candida, including C. glabrata, C. auris, C. haemulonii, C. guilliiermondii, C. krusei, C. lusitaniae, C. kefyr, Yarrowia (C.) lypolitica, and C. rugose, can be resistant to the antifungals used to treat them. Further complicating treatment of Candida infection, these fungi can be difficult to identify with standard laboratory methods and can be misidentified in labs without specific technology. Misidentification may lead to inappropriate management of the infections.

Those at risk of contracting Candida infections include patients who have been hospitalized in a healthcare facility a long time, have a central venous catheter, or other lines or tubes entering their body, have previously received antibiotics or antifungal medications, have recently had surgery, have a weakened immune system, have kidney failure or are on hemodialysis, or have diabetes. Individuals can carry the fungi on their body, even if it is not making them sick, which is called colonization. When individuals in hospitals and nursing homes are colonized, the fungi can spread from their bodies to other individuals or nearby objects. Infections have been found in individuals of all ages, from preterm infants to the elderly.

Healthcare facilities in several countries have reported that C. auris, specifically, has been causing severe illness in hospitalized patients. In some patients, this yeast can enter the bloodstream and spread throughout the body, causing serious invasive infections. C. auris has also caused wound infections and ear infections and has been isolated from respiratory and urine specimens, but it is unclear if it causes infections in the lung or bladder. This yeast often does not respond to commonly used antifungal drugs, making infections difficult to treat.

Candida infections can also result in the formation of biofilms. A biofilm is a structured consortium associated with a living or inert abiotic surface formed by microbial cells embedded in a self-produced polymer matrix principally of polysaccharide material. Non-cellular materials such as mineral crystals, corrosion particles, clay or silt particles, or blood components, depending on the environment in which the biofilm has developed, may also be found in the biofilm matrix. Biofilm-associated cells can be differentiated from their suspended counterparts by generation of an extracellular polymeric substance (EPS) matrix, reduced growth rates, and the up- and down-regulation of specific genes. Attachment of a biofilm to a surface is a complex process regulated by diverse characteristics of the growth medium, substratum, and cell surface. An established biofilm structure comprises microbial cells and EPS, has a defined architecture, and provides an optimal environment for the exchange of genetic material between cells.

Microbial biofilms are ubiquitous in nature and can be formed on a wide variety of surfaces including living tissues and indwelling medical devices. The vast majority of the medical devices may result in biofilm infections, with central line-associated bloodstream infections, catheter-associated urinary tract infections, and ventilator-associated pneumonia being the most significant. Furthermore, biofilm-associated organisms are fundamentally different from populations of suspended (planktonic) cells. Microbes in biofilms exhibit significantly reduced susceptibility to ultraviolet light, dehydration, antimicrobials, disinfectants, and innate and adaptive host immune mechanisms as a result of the EPS covering, which can act as a protective barrier. Therefore, once a biofilm infection is established, it becomes difficult to eradicate.

In one embodiment, the present disclosure is directed to the methods and compositions of using QDs in vitro to treat fungal yeast infections. These in vitro methods take advantage of the antifungal activities of the QDs. By inhibiting, stabilizing, or preventing the growth of fungi, the in vitro methods of the disclosure are useful in clinical diagnostic tests or in cell culturing applications, in which fungal growth is undesirable.

In some embodiments, fungal infections are treated by administration of an effective amount of one or more QD species. As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus, the terms “treating” and “preventing” and the like are to be considered in their broadest context. For example, treatment does not necessarily imply that a surface is treated until an infection is completely eradicated or a host is treated until total recovery.

In some embodiments, QD species are administered in an amount effective for prophylactic and/or therapeutic purposes, wherein the growth of fungal cells or biofilms thereof is prevented, stabilized, or inhibited, or wherein fungal cells are killed. To “prevent” refers to prophylactic treatment of a surface or subject not yet infected but susceptible to, or otherwise at risk of, a particular infection. To “treat” or use for “therapeutic treatment” refers to applying or administering treatment to a surface or subject already suffering from an infection to improve a condition. As used herein, “effective amount” means the amount of a compound including QDs required to inhibit, stabilize, or prevent a fungal infection.

The term “inhibiting” and variations thereof such as “inhibition” and “inhibits” as used herein in relation to the growth of fungal cells or biofilms thereof means complete or partial inhibition of the growth of fungal cells or biofilms thereof. The inhibition may be to an extent (in magnitude and/or spatially), and/or for a time, sufficient to produce the desired effect. Inhibition may be prevention, retardation, reduction or otherwise hindrance of the growth of fungal cells or biofilms thereof. Such inhibition may be in magnitude and/or be temporal or spatial in nature. Inhibition of the growth of fungal cells or biofilms thereof by an agent (i.e. a QD or antifungal) can be assessed directly or indirectly using methods known in the art, for example, determination of viable cell numbers by plate count (colony forming units/ml or CFUs), flow-based cell counting, light and fluorescence microscopy, confocal scanning laser microscopy, fluorescent dyes and proteins, dry mass measurements, total organic carbon measurements, crystal violet assays, tetrazolium salt metabolism assays, ATP bioluminescence assays, total protein determination, and quartz crystal microbalance measurements. The growth of fungal cells or biofilms thereof can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the growth of fungal cells or biofilms thereof that have not been exposed to the agent.

In one embodiment, fungal growth on a surface is inhibited, stabilized, or prevented by contacting the surface with a fungal inhibition effective amount of one or more QD species in order to inhibit the growth of a fungi. For instance, an effective amount of one or more QD species can be mixed with the sample to inhibit, stabilize, or prevent fungal growth in the sample. In another embodiment, fungal growth is inhibited by contacting the surface of the fungi to a fungal growth inhibition effective amount of the QDs.

In some embodiments, the fungal growth inhibition effective amount of the QDs can be a concentration of about 10 μg/mL or more. In some embodiments, the fungal growth inhibition effective amount of the QDs is a concentration of about 20 μg/mL or more, 40 μg/mL or more, about 80 μg/mL or more, about 100 μg/mL or more, about 125 μg/mL or more, about 150 μg/mL or more, or about 200 μg/mL or more. For the methods described above, the surface can be an in-dwelling medical device or medical or surgical tools, devices, instruments, implements, and equipment.

In some embodiments of the present disclosure, provided are methods of applying QDs to surfaces or substrates to create bioactive surface coatings. As such, the QDs provided by certain embodiments of the present disclosure can be used as an antimicrobial particle that forms antimicrobial materials that can be used in a variety of applications, including for in-dwelling implanted medical devices, sprays/wipes for disinfecting medical equipment, bedding, or other healthcare devices, by way of non-limiting example. By antimicrobial, it is meant that the material inhibits or prevents growth of microbes, including bacteria, fungi, viruses, and other spore forming organisms. In certain embodiments, an antimicrobial material according to the present disclosure exhibits antimicrobial activity. In certain embodiments, the antimicrobial material is an antifungal material.

By way of example, the antimicrobial material may exhibit an antimicrobial activity in the presence of yeast fungi. For example, the QDs prepared in accordance with certain aspects of the present disclosure may substantially inhibit the growth of fungal cells or reduce a biofilm burden (e.g., a biofilm of yeast such as C. auris) by greater than or equal to about 50% as compared to a surface of a substrate without any antimicrobial material (comprising QDs), greater than or equal to about 60%, greater than or equal to about 70%, greater than or equal to about 75%, greater than or equal to about 80%, greater than or equal to about 85%, greater than or equal to about 90%, and in certain variations, greater than or equal to about 95% of the biofilm burden on the substrate as compared to a substrate without any antimicrobial material.

One embodiment of the applications of the composition and methods is to form a film or coating on the surface of in-dwelling medical devices to inhibit, stabilize, or prevent the development of biofilm growth. In-dwelling devices include, but are not limited to, surgical implants, prosthetic devices, artificial joints, heart valves, pacemakers, vascular grafts, vascular catheters, cerebrospinal fluid shunts, urinary catheters, and continuous ambulatory peritoneal dialysis (CAPD) catheters. QDs of the present disclosure may also be used to bathe an in-dwelling device immediately before insertion. In some embodiments, the combinations can be applied as a cleaning agent, a treatment, or impregnated into or on surgical tools and endoscopy equipment. In certain embodiments, the composition and methods disclosure are used to form a film or coating on healthcare facility surfaces to inhibit, stabilize, or prevent the development of biofilm growth.

In addition to the administration of the QDs, one or more antifungals other than the QDs can be administered to the surface. The one or more antifungals can include echinocandins such as caspofungin, micafungin, and/or anidulafungin; azoles such as fluconazole, bifonazole, butoconazole, itraconazole, clotrimazole, econazole, fenticonazole, isoconazole, luliconazole, omoconazole, oxiconazole, sertaconazole, sulconazole, ticonazole, posaconazole, ketoconazole, voriconazole, isavuconazole, albaconazole, efinaconazole, propiconazole, ravuconazole, terconazole, epoxiconazole, miconazole, and/or abafungin; polyenes such as amphotericin B, nystatin, natamycin, candicidin, filipin, hamycin, and/or rimocidin; allylamines such as terbinafine, naftifine, amorolfin, butenfine; and fluorinated pyrimidine analogs such as 5-fluorocitosine.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Candida auris (C. auris) is an emergent opportunistic fungal pathogen, originally isolated in 2009, that has caused several recent outbreaks in healthcare centers around the world with high morbidity and mortality rates, especially in immunocompromised patients. The severity of the outbreaks are due to the multi-drug resistance of C. auris, its ability to create a biofilm, which further protects the pathogen, and its ability to survive on a variety of dry and humid surfaces for an extended time. To solve this problem, the inventors proposed utilization of quantum dots as an antifungal agent to inhibit biofilm formation.

To assess the dose-response effects of QDs on preformed C. auris biofilms, the inventors used a well-established phenotypic assay.²⁵ Candida auris strain 0390 was obtained from the Centers for Disease Control and Prevention (ARBank #0390), Antibiotic Resistance Isolate Bank (CDC, Atlanta, Ga., USA). This MDR isolate is resistant to amphotericin B and azoles and exhibits decreased echinocandin sensitivity. Cryopreserved yeast cells stored as glycerol stocks in an ultra-low freezer (set at −80° C.) were propagated by streaking a loopful of yeast cells onto agar plates made of yeast-peptone-dextrose (YPD). The cells were cultured overnight as previously reported.²⁴ Briefly, C. auris was cultured into flasks (150 mL) by inoculating yeast cells in 20 mL of liquid YPD containing 1% yeast extract, 2% peptone, 2% glucose [wt/vol] at 30° C. in an orbital shaker (Thermo Fisher Scientific, Waltham, Mass., USA) at 180 rpm. After 18 hr incubation, the yeast cells were twice washed with sterile phosphate-buffered saline (PBS, Sigma-Aldrich, St. Louis, Mo.), and the final inoculum size was adjusted by hemocytometer to 1×106 resuspended cells/mL in RPMI-1640 with L-Glutamine media (Cellgro, Manassas, Va., USA) buffered with 165 mM morpholinepropanesulfonic acid (MOPS) at pH 6.9 (Thermo-Fisher Scientific, Waltham, Mass.). Resuspended cells were seeded in flat-bottom 96 well microplates, and biofilms were formed following 24 h incubation at 37° C. After biofilm formation, the biofilms were washed twice with PBS to eliminate non-adherent cells.

QDs were then assessed for their ability to inhibit biofilm formation as a function of QD concentration. QDs derived from zinc oxide, zinc sulfide, silicon, and carbon were synthesized by methods known in the art.²⁰⁻²³ Synthesized QDs were harvested from solvent and redispersed in water. Varying concentrations of the QDs were added to the yeast cells in the 96 well microplates, and the plates were covered with parafilm and incubated for an additional 24 h. Subsequently, the plates were carefully washed twice with PBS to eliminate non-adherent cells.

To test the efficacy of the nanoparticle preparations on biofilm formation, the biofilms were quantified using the tetrazolium salt (2,3-Bis-(2-Methoxy-4-Nitro-5-Sulfophenyl)-2H-Tetrazolium-5-Carboxanilide [XTT]) reduction assay. All tests were performed in duplicate in independent experiments and were repeated at least three times. The half maximal inhibitory concentration (IC₅₀) was calculated based on dose-response fits calculated using a four-parameter Hill equation using SigmaPlot software (version 10.0, Systat Software, Inc., San Jose, Calif.). The IC₅₀ of each species of QD tested is shown below in Table 2.

TABLE 2
Different species of QDs and their corresponding
IC₅₀ against preformed C. auris strain 0390 biofilms
as calculated based on dose-dependent experiments.

| | Half maximal inhibitory concentration |
|---|---|
| Quantum Dot | (IC₅₀)(μg/ml) |
| Zinc Oxide | 70 |
| Zinc Sulfide | 65 |
| Silicon | 125 |
| Carbon | 57 |

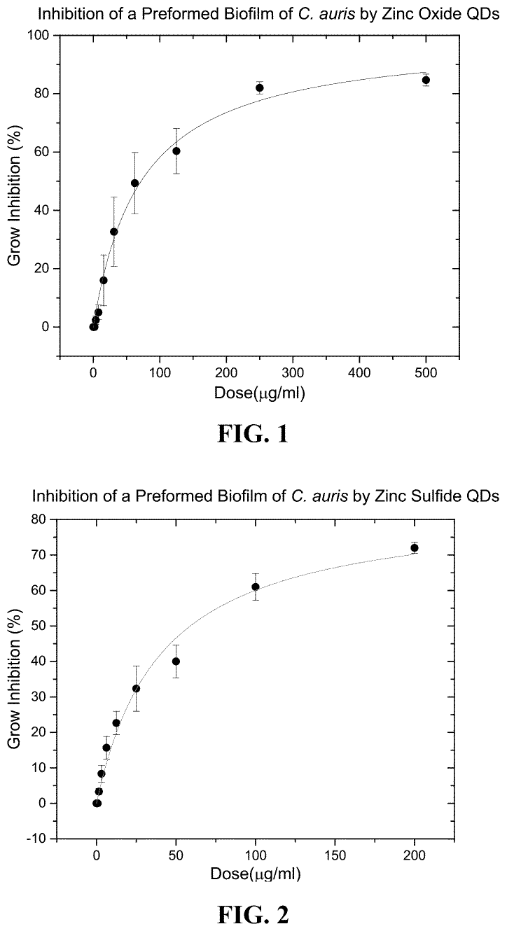

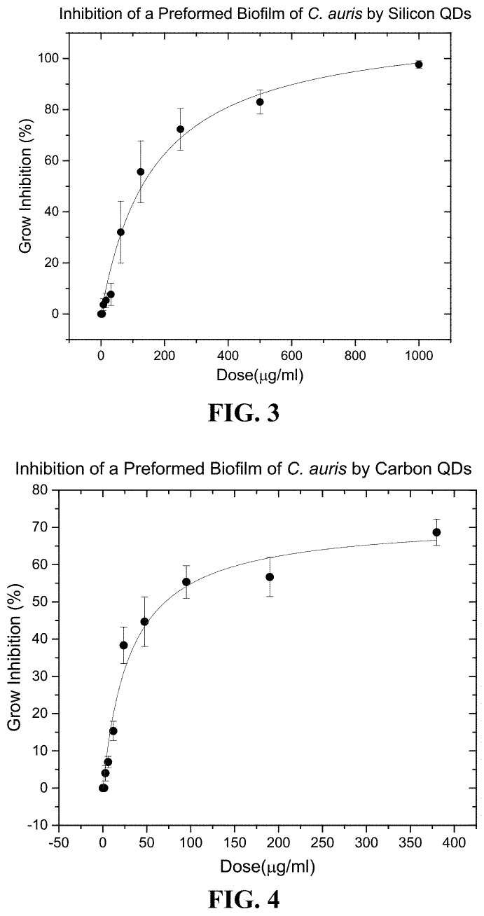

The aforementioned tests confirmed dose-dependent inhibition of C. auris biofilm formation. The IC50 was calculated to be 70 μg/mL for zinc oxide QDs (FIG. 1 and Table 1) and 65 μg/mL for zinc sulfide QDs (FIG. 2 and Table 1). A relatively high concentration of 125 μg/mL was necessary to achieve the same 50% inhibition value for silicon QDs (FIG. 3 and Table 1). Carbon QDs exhibited the most potent inhibitory behavior with an IC₅₀ of 57 μg/mL (FIG. 4 and Table 1).

These experimental observations evince the in vitro fungicidal characteristics of from zinc oxide, zinc sulfide, silicon, and carbon QDs against preformed C. auris biofilms, which are known to be resistant to most antifungal agents. Furthermore, the concentration required to achieve half-maximum inhibition are comparable among the tested QDs despite their having different elemental bases. This is the first study to effectively demonstrate the antifungal activity of QDs in vitro against Candida auris biofilms. However, the mechanism by which inhibition is achieved by the QDs still remains to be elucidated.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Satoh K, Makimura K, Hasumi Y, Nishiyama Y, Uchida K, Yamaguchi H. Candida auris sp. nov., a novel ascomycetous yeast isolated from the external ear canal of an inpatient in a Japanese hospital. Microbiol Immunol. 2009; 53(1):41-44. doi:10.1111/j.1348-0421.2008.00083.x
2. Nett JE. Candida auris: An emerging pathogen “incognito”? PLoS Pathog. 2019; 15(4):6-11. doi:https://doi.org/10.1371/journal.ppat.1007638
3. Haghani I, Ahangarkani F, Badali H, et al. Candida auris otomycosis in Iran and review of recent literature. 2019; (December 2018):101-105. doi:10.1111/myc.12886
4. Hagen F, Randhawa H S, Singh P K, et al. New Clonal Strain of Candida auris, Delhi, India. Emerg Infect Dis. 2013; 19(10):1670-1673. doi:10.3201/eid1910.130393
5. Calvo B, Melo ASA, Perozo-Mena A, et al. First report of Candida auris in America: Clinical and microbiological aspects of 18 episodes of candidemia. J Infect. 2016; 73(4):369-374. doi:10.1016/j.jinf2016.07.008
6. Vallabhaneni S, Kallen A, Tsay S, et al. Investigation of the First Seven Reported Cases of Candida auris, a Globally Emerging Invasive, Multidrug-Resistant Fungus—United States, May 2013-August 2016. Am J Transplant. 2017; 17(1):296-299. doi:10.1111/ajt.14121
7. Chowdhary A, Sharma C, Meis J F. Candida auris: A rapidly emerging cause of hospital-acquired multidrug-resistant fungal infections globally. PLOS Pathog. 2017; 13(5):e1006290. doi:10.1371/journal.ppat.1006290
8. Lamoth F, Kontoyiannis D P. The Candida auris alert: Facts and perspectives. J Infect Dis. 2018; 217(4):516-520. doi:10.1093/infdis/jix597
9. Chad J. Johnson, J. Muse Davis, Anna Huttenlocher, John F. Kernien J E N. Emerging Fungal Pathogen Candida auris Evades Neutrophil Attack. MBio. 2018; 9(4):e01403-18.
10. Donskey C, Cadnum J, Piedrahita C, Jencson A, Aaron S, Mahmoud G. Environmental Surfaces in Healthcare Facilities Are a Potential Source for Dissemination of Candida Species. Open Forum Infect Dis. 2017; 4(suppl_1):S192-S192. doi: 10.1093/ofid/ofx163.364
11. Sherry L, Ramage G, Kean R, et al. Biofilm-forming capability of highly virulent, multidrug-resistant Candida auris. Emerg Infect Dis. 2017; 23(2):328-331. doi:10.3201/eid2302.161320
12. Zucker H, Quinn M, Kallen A, et al. Candida auris in Healthcare Facilities, New York, USA, 2013-2017. Emerg Infect Dis. 2018; 24(10):1816-1824. doi:10.3201/eid2410.180649
13. Bhattacharya S, Holowka T, Orner E P, Fries B C. Gene Duplication Associated with Increased Fluconazole Tolerance in Candida auris cells of Advanced Generational Age. Sci Rep. 2019; 9(1):5052. doi:10.1038/s41598-019-41513-6
14. Ledwoch K, Maillard J-Y. Candida auris Dry Surface Biofilm (DSB) for Disinfectant Efficacy Testing. Materials (Basel). 2018; 12(1):18. doi:10.3390/ma12010018
15. Chowdhary A, Prakash A, Sharma C, et al. A multicentre study of antifungal susceptibility patterns among 350 Candida auris isolates (2009-17) in India: Role of the ERG11 and FKS1 genes in azole and echinocandin resistance. J Antimicrob Chemother. 2018; 73(4):891-899. doi:10.1093/jac/dkx480
16. Welsh R M, Bentz M L, Shams A, et al. Survival, Persistence, and Isolation of the Emerging Multidrug-Resistant Pathogenic Yeast Candida auris on a Plastic Health Care Surface. J Clin Microbiol. 2017; 55(10):2996-3005.
17. Sun H, Gao N, Dong K, Ren J, Qu X. Graphene Quantum Dots-Band-Aids Used for Wound Disinfection. 2014; (6):6202-6210. doi:10.1021/nn501640q
18. Harroun S G, Lai J Y, Huang C C, Tsai S K, Lin H J. Reborn from the Ashes: Turning Organic Molecules to Antimicrobial Carbon Quantum Dots. ACS Infect Dis. 2017; 3(11):777-779. doi:10.1021/acsinfecdis.7b00150
19. Lara H H, Urbina D G R, Pierce C, Ribot J L L, Jiménez M J A, Yacaman M J. Effect of silver nanoparticles on Candida albicans biofilms: an ultrastructural study. J Nanobiotechnology. 2015:1-12. doi:10.1186/s12951-015-0147-8
20. Rodriguez-Paéz J., Caballero a., Villegas M, Moure C, Duran P, Fernandez J. Controlled precipitation methods: formation mechanism of ZnO nanoparticles. J Eur Ceram Soc. 2001; 21(7):925-930. doi:10.1016/S0955-2219(00)00283-1
21. Li H, He X, Kang Z, et al. Water-soluble fluorescent carbon quantum dots and photocatalyst design. Angew Chemie—Int Ed. 2010; 49(26):4430-4434. doi:10.1002/anie.200906154
22. Wu S, Dou J, Zhang J, Zhang S. A simple and economical one-pot method to synthesize high-quality water soluble CdTe QDs. J Mater Chem. 2012; 22(29):14573-14578. doi:10.1039/c2jm31409f
23. Shahi A K, Pandey B K, Singh B P, Gupta B K, Singh S, Gopal R. Photo physical studies of PVP arrested ZnS quantum dots. Electron Mater Lett. 2017; 13(2):160-167. doi:10.1007/s13391-017-6132-7
24. Wall G, Chaturvedi A K, Wormley F L. Screening a Repurposing Library for Inhibitors of Multidrug-Resistant Candida auris Identifies Ebselen as a Repositionable. 2018; 62(10):1-13.
25. Lara H H, Romero-Urbina D G, Pierce C, Lopez-Ribot J L, Arellano-Jimenez M J, Jose-Yacaman M. Effect of silver nanoparticles on Candida albicans biofilms: An ultrastructural study. J Nanobiotechnology. 2015; 13(1): 1-13. doi:10.1186/s12951-015-0147-8

1. A method for treating a microbial infection on a surface, the method comprising applying to the surface in need of such treatment a composition comprising an effective amount of one or more quantum dot species.

2. The method of claim 1, wherein the effective amount of the one or more quantum dot species comprises an amount effective to inhibit, stabilize, or prevent growth of fungal cells or formation of biofilms thereof.

3. The method of claim 1 or 2, wherein inhibition by the one or more quantum dot species reduces growth of fungal cells or formation of biofilms thereof by greater than or equal to about 50% compared to the growth of fungal cells or biofilms thereof that have not been exposed to the one or more quantum dot species.

4. The method of claim 1 or 2, wherein inhibition by the one or more quantum dot species reduces growth of fungal cells or formation of biofilms thereof by greater than or equal to about 70% compared to the growth of fungal cells or biofilms thereof that have not been exposed to the one or more quantum dot species.

5. The method of any one of claims 1 to 4, wherein the quantum dots have a maximum dimension of less than or equal to about 20 nm.

6. The method of any one of claims 1 to 5, wherein the quantum dots have a maximum dimension of less than or equal to about 10 nm.

7. The method of any one of claims 1 to 5, wherein the one or more quantum dot species comprise a quantum dot comprised of a main group IV element.

8. The method of claim 7, wherein the main group IV element comprises silicon or carbon.

9. The method of any one of claims 1 to 5, wherein the one or more quantum dot species comprise a quantum dot comprised of an alloy.

10. The method of claim 9, wherein the alloy comprises a transition metal group II element and a main group VI element.

11. The method of claim 10, wherein the transition metal group II element comprises zinc or cadmium.

12. The method of claim 10, wherein the main group VI element comprises oxygen, sulphur, selenium, or tellurium.

13. The method of any one of claims 1 to 12, wherein the concentration of the one or more quantum dot species applied is between about 10 μg/mL and about 200 μg/mL.

14. The method of any one of claims 1 to 12, wherein the concentration of the one or more quantum dot species applied is between about 25 μg/mL and about 150 μg/mL.

15. The method of any one of claims 1 to 14, wherein the microbial infection is a fungal infection caused by yeast.

16. The method according to claim 15, wherein the yeast are of the genus Candida.

17. The method of claim 16, wherein the yeast is C. glabrata, C. auris, C. haemulonii, C. guilliiermondii, C. krusei, C. lusitaniae, C. kefyr, Yarrowia (C.) lypolitica, C. rugose, or a combination thereof.

18. The method of any one of claims 1 to 17, wherein the surface is an in-dwelling medical device.

19. The method of claim 18, wherein the in-dwelling medical device is a surgical implant, prosthetic device, artificial joint, heart valve, pacemaker, vascular graft, vascular catheter, cerebrospinal fluid shunt, urinary catheter, or continuous ambulatory peritoneal dialysis catheter.

20. The method of claim 18 or 19, wherein the one or more quantum dot species are applied to the in-dwelling medical device immediately before insertion.

21. The method of any one of claims 1 to 17, wherein the surface comprises medical or surgical tools, devices, instruments, implements, and equipment.

22. The method of any one of claims 1 to 21, further comprising applying the one or more quantum dot species either sequentially or simultaneously with one or more additional therapeutic agents.

23. The method of claim 22, wherein the one or more additional therapeutic agents is an anti-fungal agent or an anti-yeast agent.

24. The method of claim 23, wherein the anti-fungal or anti-yeast agent comprises an echinocandin, azole, polyene, allylamine, fluorinated pyrimidine analog, or a combination thereof.

25. An antimicrobial material comprising a bioactive coating comprising an effective amount of the one or more quantum dot species, wherein each quantum dot exhibits antimicrobial activity in the presence of a microbe.

26. The antimicrobial material of claim 25, wherein the microbe comprises fungal yeast.

27. The antimicrobial material of claim 26, wherein the yeast are of the genus Candida.

28. The antimicrobial material of claim 27, wherein the yeast is C. glabrata, C. auris, C. haemulonii, C. guilliiermondii, C. krusei, C. lusitaniae, C. kefyr, Yarrowia (C.) lypolitica, C. rugose, or a combination thereof.

29. The antimicrobial material of any one of claims 25 to 28, wherein the antimicrobial material reduces the growth of fungal cells or formation of biofilms thereof by greater than or equal to about 50% as compared to a surface without the bioactive coating.

30. The antimicrobial material of any one of claims 25 to 28, wherein the antimicrobial material reduces the growth of fungal cells or formation of biofilms thereof by greater than or equal to about 70% as compared to a surface without the bioactive coating.

31. The antimicrobial material of any one claims 25 to 30, wherein the quantum dots have a maximum dimension of less than or equal to about 20 nm.

32. The antimicrobial material of any one of claims 25 to 31, wherein the quantum dots have a maximum dimension of less than or equal to about 10 nm.

33. The antimicrobial material of any one of claims 25 to 31, wherein the one or more quantum dot species comprise a quantum dot comprised of a main group IV element.

34. The antimicrobial material of claim 33, wherein the main group IV element comprises silicon or carbon.

35. The antimicrobial material of any one of claims 25 to 31, wherein the one or more quantum dot species comprise a quantum dot comprised of an alloy.

36. The antimicrobial material of claim 35, wherein the alloy comprises a transition metal group II element and a main group VI element.

37. The antimicrobial material of claim 36, wherein the transition metal group II element comprises zinc or cadmium.

38. The antimicrobial material of claim 36, wherein the main group VI element comprises oxygen, sulphur, selenium, or tellurium.

39. The antimicrobial material of any one of claims 25 to 38, wherein the material is used to coat an in-dwelling medical device.

40. The antimicrobial material of claim 39, wherein the in-dwelling medical device is a surgical implant, prosthetic device, artificial joint, heart valve, pacemaker, vascular graft, vascular catheter, cerebrospinal fluid shunt, urinary catheter, or continuous ambulatory peritoneal dialysis catheter.

41. The antimicrobial material of any one of claims 25 to 38, wherein the material is used to coat medical or surgical tools, devices, instruments, implements, and equipment.

42. A composition of one or more quantum dot species, wherein the composition is effective to inhibit, stabilize, or prevent growth of fungal cells or formation of biofilms thereof.

43. The composition of claim 42, wherein inhibition by the one or more quantum dot species reduces growth of fungal cells or formation of biofilms thereof by greater than or equal to about 50%.

44. The composition of claim 43, wherein inhibition by the one or more quantum dot species reduces growth of fungal cells or formation of biofilms thereof by greater than or equal to about 70%.
